(19) United States
(12) Patent Application Publication
(10) Pub. No.: US 2022/0148737 A1
(43) Pub. Date: May 12, 2022

(54) SYSTEM AND METHOD FOR EVALUATING WELLNESS OF ONE OR MORE USERS

(71) Applicant: Creative Choice Inc., Palm Beach Gardens, FL (US)

(72) Inventor: Dilip Barot, Palm Beach Gardens, FL (US)

(21) Appl. No.: 17/580,676

(22) Filed: Jan. 21, 2022

Related U.S. Application Data
(63) Continuation-in-part of application No. 16/580,227, filed on Sep. 24, 2019, now abandoned.

(51) Int. Cl. G16H 50/30; G16H 50/20

(52) U.S. Cl. CPC G16H 50/30; G16H 50/20

(57) ABSTRACT

A system and method for evaluating wellness of one or more users is disclosed. The method includes receiving a request from one or more user devices to evaluate wellness of one or more users and determining a set of wellness parameters corresponding to each of one or more wellness pillars. The method further includes generating a pillar score for each of the one or more wellness pillars and generating an overall wellness score of the one or more users. Further, the method includes determining level of wellness of the one or more users and outputting the determined set of wellness parameters corresponding to each of the one or more wellness pillars, the generated pillar score for each of the one or more wellness pillars, the generated wellness score and the determined level of wellness on user interface screens of the one or more user devices.

### CROSS-REFERENCE TO RELATED APPLICATIONS

This application is a continuation in part of a non-provisional patent application filed in the U.S. having patent application Ser. No. 16/580,227 filed on Sep. 24, 2019 and titled “SYSTEM AND METHOD TO OFFER WELLNESS PROGRAMS”.

### FIELD OF INVENTION

Embodiments of the present disclosure relate to a health and wellness system and more particularly relates to a system and a method for evaluating wellness of one or more users.

### BACKGROUND

Wellness is an active lifestyle incorporating multiple components which affects physical, mental and social wellbeing. With the advancements in technology and ever-increasing demands for greater productivity, people are suffering from excessive stress in professional and personal lives affecting their wellness. Further, people are also facing multiple issues, such as high blood pressure, heart problems, obesity, headaches, depression and anxiety, gastrointestinal problems, accelerated ageing and the like. Thus, people are looking for ways to find balance in their fast-paced lives. Generally, people are resorting to different sorts of medication for treating lifestyle related illnesses and achieving wellness. However, it has been proven that the medication is not an effective method to cure the problem at hand.

Conventionally, there are multiple systems for evaluating wellness of one or more users. However, conventional systems fail to consider multiple pillars of wellness, such as mindfulness, relaxation, sleep and the like while evaluating the wellness of the one or more users. Thus, the conventional systems are not accurate and precise on an individual's overall wellness. Further, the conventional systems also fail to predict possible health conditions, such as heart attack, diabetes and the like, of the one or more users and time of occurrence of the possible health conditions. Thus, the one or more users lose chance of receiving early treatment and changing their lifestyle to allay or prevent the occurrence of the possible health conditions.

Hence, there is a need for an improved system and method for evaluating wellness of one or more users, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computing system for evaluating wellness of one or more users is disclosed. The computing system includes one or more hardware processors and a memory coupled to the one or more hardware processors. The memory includes a plurality of modules in the form of programmable instructions executable by the one or more hardware processors. The plurality of modules include a request receiver module configured to receive a request from one or more user devices to evaluate wellness of one or more users. The request includes: name, address, weight, height, glucose, cholesterol, triglycerides, gender, age and experience level of the one or more users. The plurality of modules also include a parameter determination module configured to determine a set of wellness parameters corresponding to each of one or more wellness pillars based on the received request and a set of predefined rules by using a trained wellness evaluation based Artificial Intelligence (AI) model. The one or more wellness pillars include: relaxation pillar, fitness pillar, mindfulness pillar, nutrition pillar and sleep pillar. The plurality of modules includes a pillar score generation module configured to generate a pillar score for each of the one or more wellness pillars based on the received request, the set of predefined rules, the determined set of wellness parameters corresponding to each of the one or more wellness pillars and a predefined pillar weightage by using the trained wellness evaluation based AI model. Further, the plurality of modules includes a wellness score generation module configured to generate a wellness score of the one or more users based on the generated pillar score of each of the one or more wellness pillars, the received request, the set of predefined rules and the predefined pillar weightage by using the trained wellness evaluation based AI model. The plurality of modules also include a wellness level determination module configured to determine level of wellness of the one or more users based on the generated wellness score, predefined wellness information and the received request by using the trained wellness evaluation based AI model. Furthermore, the plurality of modules include a data output module configured to output the determined set of wellness parameters corresponding to each of the one or more wellness pillars, the generated pillar score for each of the one or more wellness pillars, the generated wellness score and the determined level of wellness on user interface screens of the one or more user devices.

In accordance with another embodiment of the present disclosure, a method for evaluating wellness of one or more users is disclosed. The method includes receiving a request from one or more user devices to evaluate wellness of one or more users. The request includes: name, address, weight, height, glucose, cholesterol, triglycerides, gender, age and experience level of the one or more users. The method also includes determining a set of wellness parameters corresponding to each of one or more wellness pillars based on the received request and a set of predefined rules by using a trained wellness evaluation based Artificial Intelligence (AI) model. The one or more wellness pillars include: relaxation pillar, fitness pillar, mindfulness pillar, nutrition pillar and sleep pillar. The method further includes generating a pillar score for each of the one or more wellness pillars based on the received request, the set of predefined rules, the determined set of wellness parameters corresponding to each of the one or more wellness pillars and a predefined pillar weightage by using the trained wellness evaluation based AI model. Further, the method includes generating a wellness score of the one or more users based on the generated pillar score of each of the one or more wellness pillars, the received request, the set of predefined rules and the predefined pillar weightage by using the trained wellness evaluation based AI model. Also, the method includes determining level of wellness of the one or more users based on the generated wellness score, predefined wellness information and the received request by using the trained wellness evaluation based AI model. Furthermore, the method includes outputting the determined set of wellness parameters corresponding to each of the one or more wellness pillars, the generated pillar score for each of the one or more wellness pillars, the generated wellness score and the determined level of wellness on user interface screens of the one or more user devices.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for evaluating wellness of one or more users, in accordance with an embodiment of the present disclosure;

FIG. 2 is a block diagram illustrating an exemplary computing system for evaluating wellness of the one or more users, in accordance with an embodiment of the present disclosure;

FIG. 3 is a process flow diagram illustrating an exemplary method for evaluating wellness of the one or more users, in accordance with an embodiment of the present disclosure; and

FIGS. 4A-4I are graphical user interface screens of dashboard of the computing system for evaluating wellness of the one or more users, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

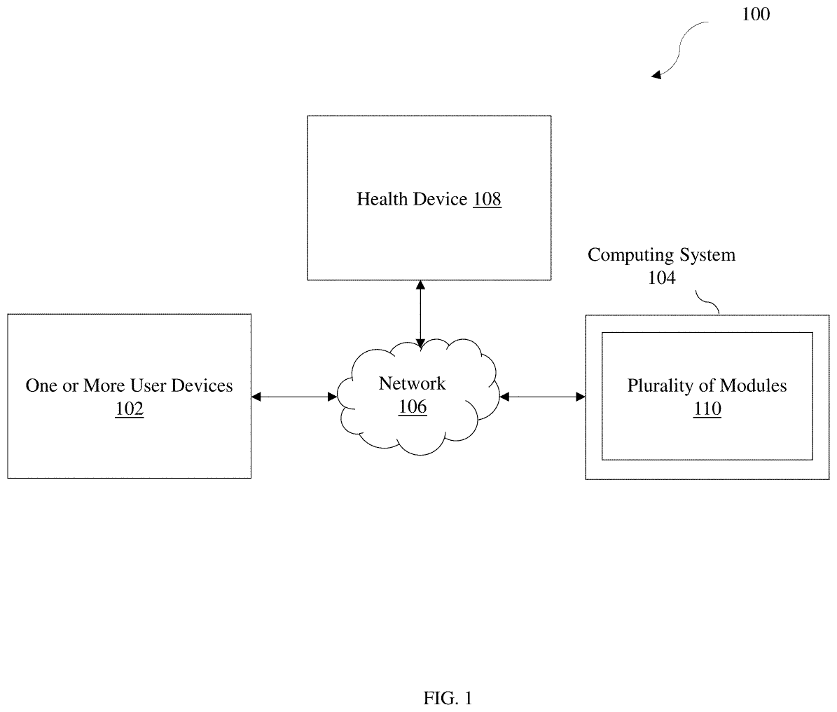

Referring now to the drawings, and more particularly to FIG. 1 through FIG. 4I, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for evaluating wellness of one or more users, in accordance with an embodiment of the present disclosure. According to FIG. 1, the computing environment 100 includes one or more user devices 102 associated with one or more users communicatively coupled to a computing system 104 via a network 106. The one or more user devices 102 are used by the one or more users to request the computing system 104 to evaluate the wellness. In an embodiment of the present disclosure, the wellness of the one or more users corresponds to one or more wellness pillars. In an exemplary embodiment of the present disclosure, the one or more wellness pillars include relaxation pillar, fitness pillar, mindfulness pillar, nutrition pillar, sleep pillar and the like. In an embodiment of the present disclosure, the request includes name, address, weight, height, glucose, cholesterol, triglycerides, gender, age, experience level of the one or more users and the like. The one or more user devices 102 are also used by the one or more users to receive information associated with the wellness of the one or more users including set of wellness parameters corresponding to each of the one or more wellness pillars, pillar score for each of the one or more wellness pillars, wellness score and level of wellness from the computing system 104. In an embodiment of the present disclosure, the set of wellness parameters corresponding to the nutrition pillar are obtained via a health device 108. In an exemplary embodiment of the present disclosure, the health device 108 may be a finger prick device. The health device 108 may be a wearable device. In another embodiment of the present disclosure, the set of wellness parameters corresponding to the nutrition pillar are obtained via a collection of bodily matter, such as stool, urine and the like. In an exemplary embodiment of the present disclosure, the one or more user devices 102 may include a laptop computer, desktop computer, tablet computer, smartphone, wearable device, smart watch and the like. Further, the network 106 may be internet or any other wireless network. The computing system 104 may be hosted on a central server, such as cloud server or a remote server.

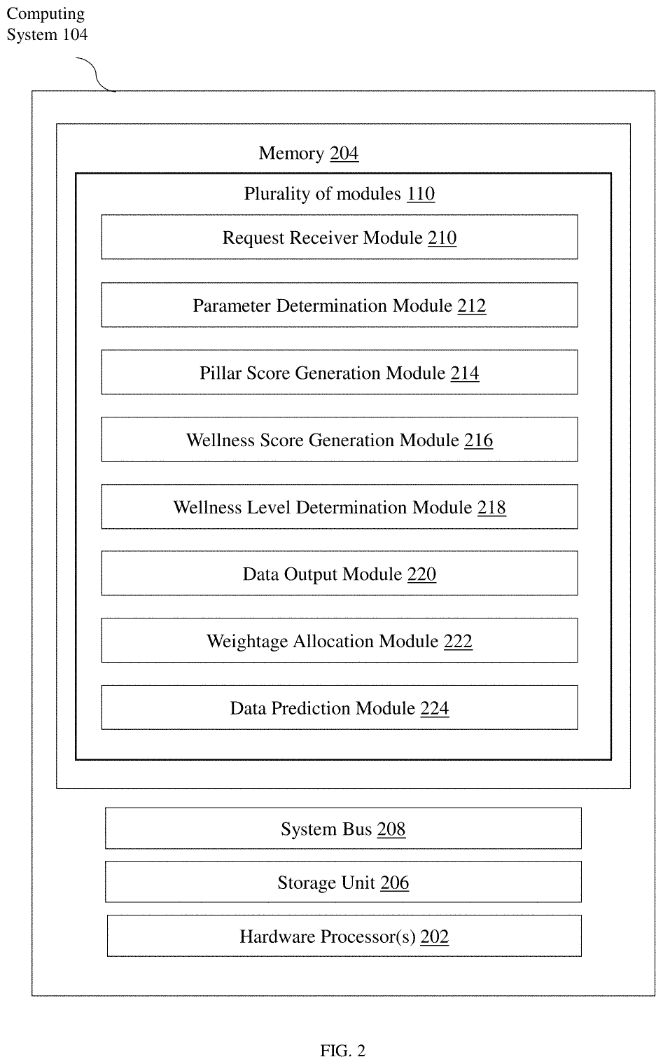

Further, the one or more user devices 102 include a local browser, a mobile application or a combination thereof. Furthermore, the one or more users may use a web application via the local browser, the mobile application or a combination thereof to communicate with the computing system 104. In an embodiment of the present disclosure, the computing system 104 includes a plurality of modules 110. Details on the plurality of modules 110 have been elaborated in subsequent paragraphs of the present description with reference to FIG. 2.

In an embodiment of the present disclosure, the computing system 104 is configured to receive the request from the one or more user devices 102 to evaluate wellness of the one or more users. Further, the computing system 104 determines the set of wellness parameters corresponding to each of the one or more wellness pillars based on the received request and a set of predefined rules by using a trained wellness evaluation based Artificial Intelligence (AI) model. The computing system 104 generate a pillar score for each of the one or more wellness pillars based on the received request, the set of predefined rules, the determined set of wellness parameters corresponding to each of the one or more wellness pillars and a predefined pillar weightage by using the trained wellness evaluation based AI model. The computing system 104 generates the wellness score of the one or more users based on the generated pillar score of each of the one or more wellness pillars, the received request, the set of predefined rules and the predefined pillar weightage by using the trained wellness evaluation based AI model. The computing system 104 determines the level of wellness of the one or more users based on the generated wellness score, predefined wellness information and the received request by using the trained wellness evaluation based AI model. Further, the computing system 104 outputs the determined set of wellness parameters corresponding to each of the one or more wellness pillars, the generated pillar score for each of the one or more wellness pillars, the generated wellness score and the determined level of wellness on user interface screens of the one or more user devices 102.

FIG. 2 is a block diagram illustrating an exemplary computing system 104 for evaluating wellness of the one or more users, in accordance with an embodiment of the present disclosure. Further, the computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a request receiver module 210, a parameter determination module 212, a pillar score generation module 214, a wellness score generation module 216, a wellness level determination module 218, a data output module 220, a weightage allocation module 222 and a data prediction module 224.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

The storage unit 206 may be a cloud storage. The storage unit 206 may store the received request, the set of wellness parameters corresponding to each of the one or more wellness pillars and the pillar score for each of the one or more wellness pillars. The storage unit 206 may also store the set of predefined rules, the predefined pillar weightage and the predefined wellness information.

The request receiver module 210 is configured to receive the request from the one or more user devices 102 to evaluate wellness of the one or more users. In an embodiment of the present disclosure, the request includes weight, height, glucose, cholesterol, triglycerides, gender, age, experience level of the one or more users and the like. In an embodiment of the present disclosure, the age of the one or more users are classified into a predefined age range. For example, the predefined age range may include 17 to 19 years, 20 to 29 years, 30 to 39 years, 40 to 49 years and the like. In an exemplary embodiment of the present disclosure, the one or more user devices 102 may include a laptop computer, desktop computer, tablet computer, smartphone, wearable device, smart watch, and the like.

The parameter determination module 212 is configured to determine the set of wellness parameters corresponding to each of the one or more wellness pillars based on the received request and the set of predefined rules by using the trained wellness evaluation based Artificial Intelligence (AI) model. In an exemplary embodiment of the present disclosure, the one or more wellness pillars include relaxation pillar, fitness pillar, mindfulness pillar, nutrition pillar, sleep pillar and the like.

The pillar score generation module 214 is configured to generate the pillar score for each of the one or more wellness pillars based on the received request, the set of predefined rules, the determined set of wellness parameters corresponding to each of the one or more wellness pillars and the predefined pillar weightage by using the trained wellness evaluation based AI model. In an exemplary embodiment of the present disclosure, the pillar score for each of the one or more wellness pillars may be 100. In an embodiment of the present disclosure, 100 is maximum or perfect wellness score. In generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module 214 determines one or more fitness parameters scores for the determined set of wellness parameters corresponding to the fitness pillar based on the received request, the set of predefined rules and the predefined pillar weightage by using the trained wellness evaluation based AI model. In an exemplary embodiment of the present disclosure, the set of wellness parameters corresponding to the fitness pillar include muscular strength, cardiovascular endurance, muscular endurance, flexibility, sit and reach, body composition, calories, cadence, distance, pace, heart rate, duration and the like. In an exemplary embodiment of the present disclosure, the muscle strength may be measured via push up test, the cardiovascular endurance may be measured via 1.5 miles run, the muscular endurance may be measured via squat test, the flexibility may be measured via sit and reach test, body composition is measured via body fat and the like. In an exemplary embodiment of the present disclosure, the body fat may be measured by using medical equipments, such as bioelectric impedance device. Further, the pillar score generation module 214 generates a fitness score based on the determined one or more fitness parameters scores for the determined set of wellness parameters, the received request, the set of predefined rules and the predefined pillar weightage by using the trained wellness evaluation based AI model. In an exemplary embodiment of the present disclosure, when the fitness pillar includes five set of wellness parameters, each of the one or more fitness parameters scores for the five set of wellness parameters may be 20.

Further, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module 214 outputs a relaxation questionnaire on the user interface screens of the one or more user devices 102. In an exemplary embodiment of the present disclosure, the relaxation questionnaire includes multiple relaxation questions for generating a relaxation score. For example, the multiple relaxation questionnaire includes in the last month, how often have you been upset because of something that happened unexpectedly, in the last month, how often have you felt that you were unable to control the important things in your life, in the last month, how often have you felt nervous and stressed, in the last month, how often have you found that you could not cope with all the things that you had to do, in the last month, how often have you felt difficulties were piling up so high that you could not overcome them, in the last month, how often have you felt confident about your ability to handle your personal problems, in the last month, how often have you felt that things were going your way, in the last month, how often have you been able to control irritations in your life, in the last month, how often have you felt that you were on top of things and the like. The pillar score generation module 214 obtains one or more responses of the one or more users on the outputted relaxation questionnaire from the one or more user devices 102. For example, the one or more responses may include never, almost never, sometimes, fairly often, very often and the like. Furthermore, the pillar score generation module 214 determines one or more relaxation questionnaire scores corresponding to the relaxation questionnaire and one or more relaxation parameters scores for the determined set of wellness parameters corresponding to the relaxation pillar based on the obtained one or more responses, the received request, the predefined wellness information, the set of predefined rules and the predefined pillar weightage by using the trained wellness evaluation based AI model. In an exemplary embodiment of the present disclosure, 10 score is provided for “never” response, 8 score is provided for “almost” response, 6 score is provided for “sometimes” response, 4 score is provided for “fairly often” response and 2 score is provided for “very often” response. The scoring may also be reversed based on one or more questions in the relaxation questionnaire. In another exemplary embodiment of the present disclosure, 2 score is provided for “never” response, 4 score is provided for “almost” response, 6 score is provided for “sometimes” response, 8 score is provided for “fairly often” response and 10 score is provided for “very often” response. In an exemplary embodiment of the present disclosure, the one or more relaxation scores are calculated by using a Perceived Stress Scale (PSS). For example, the PSS scale includes multiple questions to determine feelings and thoughts of the user during the last month. The pillar score generation module 214 generates the relaxation score based on the determined one or more relaxation questionnaire scores, the received request, the one or more relaxation parameters scores, the set of predefined rules and the predefined pillar weightage by using the trained wellness evaluation based AI model. In an exemplary embodiment of the present disclosure, the relaxation score ranging from 80-100 is considered as low stress, the relaxation score ranging from 60-80 is considered as moderate stress, the relaxation score ranging from 40-60 is considered as high stress and the relaxation score ranging from 20-40 is considered as high perceived stress.

Furthermore, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module 214 outputs a mindfulness questionnaire on the user interface screens of the one or more user devices 102. In an exemplary embodiment of the present disclosure, the mindfulness questionnaire is a short form of 15-item Five Facet Mindfulness Questionnaire (FFMQ) including multiple facets associated with observing, describing, acting with awareness, non-judging of inner experience, and non-reactivity to inner experience. In an exemplary embodiment of the present disclosure, the mindfulness questionnaire includes multiples mindfulness questions to determine a mindfulness score. For example, the multiple mindfulness questions include I don't pay attention to what I'm doing because I'm daydreaming, worrying, or otherwise distracted, I believe some of my thoughts are abnormal or bad and I shouldn't think that way, I have trouble thinking of the right words to express how I feel about things, I do jobs or tasks automatically without being aware of what I'm doing, I think some of my emotions are bad or inappropriate and I shouldn't feel them, I find myself doing things without paying attention, I tell myself I shouldn't be feeling the way I'm feeling, when I take a shower or a bath, I stay alert to the sensations of water on my body, I'm good at finding words to describe my feelings, when I have distressing thoughts or images, I “step back” and am aware of the thought or image without getting taken over by it, I notice how foods and drinks affect my thoughts, bodily sensations, and emotions, when I have distressing thoughts or images I am able just to notice them without reacting, I pay attention to sensations, such as the wind in my hair or sun on my face, even when I'm feeling terribly upset I can find a way to put it into words, when I have distressing thoughts or images I just notice them and let them go and the like. The pillar score generation module 214 obtains one or more responses of the one or more users on the outputted mindfulness questionnaire from the one or more user devices 102. For example, the one or more responses may include never, almost never, sometimes, fairly often, very often and the like. In an exemplary embodiment of the present disclosure, the user may use 1 (never or very rarely true) to 5 (very often or always true) scale to indicate relevance of each statement of the mindfulness questionnaire to the user. For example, when a statement is often true to the user, the user may select ‘4’ and when the statement is sometimes true to the user, the user may select ‘3’. The scoring may also be reversed based on one or more questions in the mindfulness questionnaire. In another exemplary embodiment of the present disclosure, the user may use 1 (very often or always true) to 5 (never or very rarely true) scale to indicate relevance of each statement of the mindfulness questionnaire to the user. Further, the pillar score generation module 214 determines one or more mindfulness questionnaire scores corresponding to the mindfulness questionnaire and one or more mindfulness parameters scores for the determined set of wellness parameters corresponding to the mindfulness pillar based on the obtained one or more responses, the received request, the predefined wellness information, the set of predefined rules and the predefined pillar weightage by using the trained wellness evaluation based AI model. In an exemplary embodiment of the present disclosure, the set of wellness parameters corresponding to the mindfulness pillar include calm time, focus time, training time and the like. Furthermore, the pillar score generation module 214 generates the mindfulness score based on the determined one or more mindfulness questionnaire scores, the received request, the one or more mindfulness parameters scores, the set of predefined rules and the predefined pillar weightage by using the trained wellness evaluation based AI model.

Further, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module 214 determines one or more nutrition parameters scores for the determined set of wellness parameters corresponding to the nutrition pillar based on the received request, the set of predefined rules and the predefined pillar weightage by using the trained wellness evaluation based AI model. In an embodiment of the present disclosure, the set of wellness parameters corresponding to the nutrition pillar are obtained via the health device 108. In an exemplary embodiment of the present disclosure, the health device 108 may be a finger prick device. The health device 108 may be a wearable device. In another embodiment of the present disclosure, the set of wellness parameters corresponding to the nutrition pillar are obtained via a collection of bodily matter, such as stool, urine and the like. In an exemplary embodiment of the present disclosure, the set of wellness parameters corresponding to the nutrition pillar comprise: Body Mass Index (BMI), glucose, total cholesterol, risk ratio, Low-Density Lipoprotein (LDL), High-Density Lipoprotein (HDL), triglycerides, gut microbiome analysis, stress analysis, immune system health, biological age and the like. For example, the set of predefined rules corresponding to the nutrition pillar are: BMI=weight divided by height squared=over 25 BMI no points Vs under 25 BMI=16.67 points, glucose=non fasting under 140 fasting under 100=16.67 points, total cholesterol—in normal range add full credit, if over range no points are added under 200=16.67 points, LDL—under 100=16.67 points, HDL—over 60=16.67 points, triglycerides—less than 150=16.67 points and the like. Furthermore, the pillar score generation module 214 generates a nutrition score based on the determined one or more nutrition parameter scores for the determined set of wellness parameters, the received request, the set of predefined rules and the predefined pillar weightage by using the trained wellness evaluation based AI model. In an exemplary embodiment of the present disclosure, when the nutrition pillar includes six set of wellness parameters, each of the one or more nutrition parameters scores for the six set of wellness parameters may be 16.67.

In an embodiment of the present disclosure, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module 214 determines one or more sleep parameters scores for the determined set of wellness parameters corresponding to the sleep pillar based on the received request, the set of predefined rules and the predefined pillar weightage by using the trained wellness evaluation based AI model. In an exemplary embodiment of the present disclosure, the set of wellness parameters corresponding to the sleep pillar include total time in bed, sleep latency, readiness, activity, sleep waking, actual sleep time, wakefulness, sleep efficiency, efficiency resting heart rate, Heart Rate Variability (HRV), respiration rate, body temperature and the like. For example, the set of predefined rules for calculating sleep efficiency is 480 (total minutes in bed)−30 (minutes to fall asleep)−0 (minutes awake during the night)=450 (actual sleep time in minutes) i.e., 450/480=0.9375×100=93.75% sleep efficiency. Furthermore, the pillar score generation module 214 generates a sleep score based on the determined one or more sleep parameter scores for the determined set of wellness parameters, the received request, the set of predefined rules and the predefined pillar weightage by using the trained wellness evaluation based AI model.

In an embodiment of the present disclosure, each of the set of wellness parameters corresponding to the one or more wellness pillars are categorized into one or more wellness categories based on a set of parameter scores. In an exemplary embodiment of the present disclosure, the one or more relaxation categories include elite, advanced, intermediate, beginner, new and the like. In an exemplary embodiment of the present disclosure, the set of parameter scores include the one or more fitness parameters scores, the one or more relaxation questionnaire scores, the one or more relaxation parameters scores, the one or more mindfulness questionnaire scores, the one or more mindfulness parameters scores, the one or more nutrition parameters scores and the one or more sleep parameters scores.

The wellness score generation module 216 is configured to generate a wellness score of the one or more users based on the generated pillar score of each of the one or more wellness pillars, the received request, the set of predefined rules and the predefined pillar weightage by using the trained wellness evaluation based AI model. In generating the wellness score of the one or more users based on the generated pillar score of each of the one or more wellness pillars, the received request, the set of predefined rules and the predefined pillar weightage by using the trained wellness evaluation based AI model, the wellness score generation module 216 correlates the pillar score of each of the one or more wellness pillars, the received request, the set of predefined rules and the predefined pillar weightage by using the trained wellness evaluation based AI model. In an embodiment of the present disclosure, the pillar score of each of the one or more wellness pillars include the fitness score, the relaxation score, the nutrition score, the mindfulness score and the sleep score. Further, the wellness score generation module 216 generates the wellness score of the one or more users based on the result of correlation.

The wellness level determination module 218 is configured to determine level of wellness of the one or more users based on the generated wellness score, predefined wellness information and the received request by using the trained wellness evaluation based AI model. In an exemplary embodiment of the present disclosure, the level of wellness of the one or more users include elite, advanced, intermediate, beginner, new and the like.

The data output module 220 is configured to output the determined set of wellness parameters corresponding to each of the one or more wellness pillars, the generated pillar score for each of the one or more wellness pillars, the generated wellness score and the determined level of wellness on user interface screens of the one or more user devices 102. In an exemplary embodiment of the present disclosure, the determined set of wellness parameters corresponding to each of the one or more wellness pillars, the generated pillar score for each of the one or more wellness pillars, the generated wellness score and the determined level of wellness are outputted on the user interface screens of the one or more user devices 102 by using Transport layer Security (TLS) 1.2.

The weightage allocation module 222 is configured to receive one or more wellness preferences from the one or more user devices 102. In an exemplary embodiment of the present disclosure, the one or more wellness preferences include weight loss, weight gain, stress management, anxiety management, sleep management and the like. Further, the weightage allocation module 222 dynamically allocates one or more parameter weightages to the set of wellness parameters of each of the one or more wellness pillars based on the received one or more wellness preferences, the received request and the predefined wellness information by using the trained wellness evaluation based AI model. The one or more parameter weightages include one or more maximum parameter scores for the set of wellness parameters of each of the one or more wellness pillars. In an exemplary embodiment of the present disclosure, each of the one or more parameter weightages may not be less than 1. In an embodiment of the present disclosure, the set of parameter scores for the set of wellness parameters corresponding to each of the one or more wellness pillars are generated based on the allocated one or more parameter weightages. The set of parameter scores for the set of wellness parameters may be equal or less than the one or more maximum parameter scores. Furthermore, the weightage allocation module 222 dynamically allocates a pillar weightage to each of the one or more wellness pillars based on the received one or more wellness preferences, the received request and the predefined wellness information by using the trained wellness evaluation based AI model. In an embodiment of the present disclosure, the pillar weightage includes maximum pillar score of each of the one or more wellness pillars. In an exemplary embodiment of the present disclosure, combined pillar weightage of the one or more fitness pillar may not exceed 100. In another exemplary embodiment of the present disclosure, the combined pillar weightage of the one or more wellness pillars may exceed 100. In an embodiment of the present disclosure, the pillar score for each of the one or more wellness pillars is generated based on the allocated pillar weightage. The pillar score for each of the one or more wellness pillars may be equal or less than the maximum pillar score. In an exemplary embodiment of the present disclosure, the one or more parameter weightages and the pillar weightage are in percentage form. In an embodiment of the present disclosure, the set of parameter scores generated based on the one or more parameter weightages of a specific pillar may again be quantified based on the pillar weightage of the specific pillar to generate the pillar score.

In an embodiment of the present disclosure, the weightage allocation module 222 may also receive the pillar weightage for each of the one or more wellness pillars and the one or more parameter weightages for the set of wellness parameters of each of the one or more wellness pillars from the one or more user devices 102. In another embodiment of the present disclosure, the pillar weightage for each of the one or more wellness pillars may be equally distributed. For example, the sleep pillar has the pillar weightage of 20, the nutrition pillar has the pillar weightage of 20, the fitness pillar has the pillar weightage of 20, the mindfulness pillar has the pillar weightage of 20 and the relaxation pillar has the pillar weightage of 20. Further, combination of the one or more parameter weightages corresponding to each of the one or more wellness pillars may not exceed the pillar weightage of each of the one or more wellness pillars. For example, if the pillar weightage of the sleep pillar is 20, then the combination of the one or more parameter weightages for the set of wellness parameters of the sleep pillar, such as sleep, readiness, heart rate variability, activity and the like may not exceed 20. In an embodiment of the present disclosure, the one or more parameter weightages for the set of wellness parameters may be equally distributed. The weightage allocation module 222 allocates the received pillar weightage to each of the one or more wellness pillars. Furthermore, the weightage allocation module 222 allocates the received one or more parameter weightages to the set of wellness parameters of each of the one or more wellness pillars.

The data prediction module 224 is configured to determine if the determined level of wellness of the one or more users is below a predefined threshold wellness level. Further, the data prediction module 224 determines one or more root causes for the determined level of wellness based on the determined level of wellness, the set of parameter scores and the predefined wellness information by using the trained wellness evaluation based AI model upon determining that the determined level of wellness is below the predefined threshold wellness level. In an exemplary embodiment of the present disclosure, the one or more root causes may be less sleep, high cholesterol, high sugar, high blood pressure and the like. The data prediction module 224 predicts one or more possible health conditions of the one or more users based on the determined one or more root causes, the determined level of wellness, the set of parameter scores and the predefined wellness information by using the trained wellness evaluation based AI model. For example, the one or more possible health conditions may be heart attack, diabetes and the like. Furthermore, the data prediction module 224 predicts time of occurrence of the predicted one or more possible conditions based on the determined one or more root causes, the determined level of wellness, the set of parameter scores and the predefined wellness information by using the trained wellness evaluation based AI model. In an embodiment of the present disclosure, the determined one or more root causes, the predicted one or more possible health conditions and the predicted time of occurrence of the predicted one or more possible conditions are outputted on the user interface screens of the one or more user devices 102.

In operation, the one or more users use their credentials to login into the computing system 104. In an exemplary embodiment of the present disclosure, the credentials are outputted on the user interface screens of the one or more user devices 102 via email, the mobile application, the web application and the like. Further, the computing system 104 receives the request from the one or more user devices 102 to evaluate wellness of the one or more users. The computing system 104 determines the set of wellness parameters corresponding to each of the one or more wellness pillars based on the received request and the set of predefined rules by using the trained wellness evaluation based Artificial Intelligence (AI) model. Furthermore, the computing system 104 generates the pillar score for each of the one or more wellness pillars based on the received request, the set of predefined rules, the determined set of wellness parameters corresponding to each of the one or more wellness pillars and the predefined pillar weightage by using the trained wellness evaluation based AI model. The computing system 104 generates the wellness score of the one or more users based on the generated pillar score of each of the one or more wellness pillars, the received request, the set of predefined rules and the predefined pillar weightage by using the trained wellness evaluation based AI model. Further, the computing system 104 determines the level of wellness of the one or more users based on the generated wellness score, the predefined wellness information and the received request by using the trained wellness evaluation based AI model. The computing system 104 outputs the determined set of wellness parameters corresponding to each of the one or more wellness pillars, the generated pillar score for each of the one or more wellness pillars, the generated wellness score and the determined level of wellness on user interface screens of the one or more user devices 102.

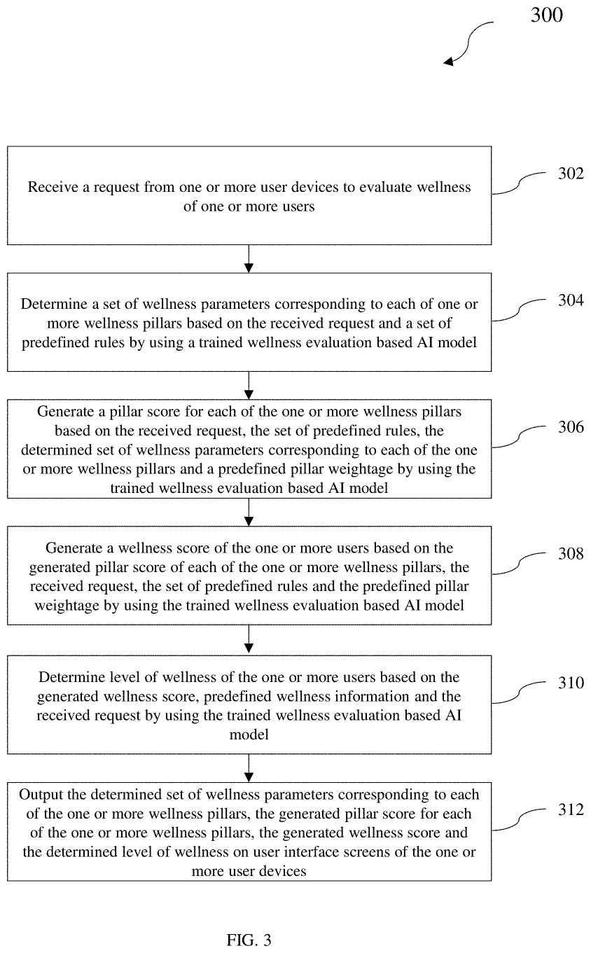

FIG. 3 is a process flow diagram illustrating an exemplary method for evaluating wellness of one or more users, in accordance with an embodiment of the present disclosure. At step 302, a request is received from one or more user devices 102 to evaluate wellness of one or more users. In an embodiment of the present disclosure, the request includes weight, height, glucose, cholesterol, triglycerides, gender, age, experience level of the one or more users and the like. In an embodiment of the present disclosure, the age of the one or more users are classified into a predefined age range. For example, the predefined age range may include 17 to 19 years, 20 to 29 years, 30 to 39 years, 40 to 49 years and the like. In an exemplary embodiment of the present disclosure, the one or more user devices 102 may include a laptop computer, desktop computer, tablet computer, smartphone, wearable device, smart watch, and the like.

At step 304, a set of wellness parameters corresponding to each of one or more wellness pillars are determined based on the received request and a set of predefined rules by using a trained wellness evaluation based Artificial Intelligence (AI) model. In an exemplary embodiment of the present disclosure, the one or more wellness pillars include relaxation pillar, fitness pillar, mindfulness pillar, nutrition pillar, sleep pillar and the like.

At step 306, a pillar score for each of the one or more wellness pillars is generated based on the received request, the set of predefined rules, the determined set of wellness parameters corresponding to each of the one or more wellness pillars and a predefined pillar weightage by using the trained wellness evaluation based AI model. In an exemplary embodiment of the present disclosure, the pillar score for each of the one or more wellness pillars may be 100. In an embodiment of the present disclosure, 100 is maximum or perfect wellness score. In generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the method 300 includes determining one or more fitness parameters scores for the determined set of wellness parameters corresponding to the fitness pillar based on the received request, the set of predefined rules and the predefined pillar weightage by using the trained wellness evaluation based AI model. In an exemplary embodiment of the present disclosure, the set of wellness parameters corresponding to the fitness pillar include muscular strength, cardiovascular endurance, muscular endurance, flexibility, sit and reach, body composition, calories, cadence, distance, pace, heart rate, duration and the like. In an exemplary embodiment of the present disclosure, the muscle strength may be measured via push up test, the cardiovascular endurance may be measured via 1.5 miles run, the muscular endurance may be measured via squat test, the flexibility may be measured via sit and reach test, body composition is measured via body fat and the like. In an exemplary embodiment of the present disclosure, the body fat may be measured by using medical equipments, such as bioelectric impedance device. Further, the method 300 includes generating a fitness score based on the determined one or more fitness parameters scores for the determined set of wellness parameters, the received request, the set of predefined rules and the predefined pillar weightage by using the trained wellness evaluation based AI model. In an exemplary embodiment of the present disclosure, when the fitness pillar includes five set of wellness parameters, each of the one or more fitness parameters scores for the five set of wellness parameters may be 20.

Further, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the method 300 includes outputting a relaxation questionnaire on the user interface screens of the one or more user devices 102. In an exemplary embodiment of the present disclosure, the relaxation questionnaire includes multiple relaxation questions for generating a relaxation score. For example, the multiple relaxation questionnaire includes in the last month, how often have you been upset because of something that happened unexpectedly, in the last month, how often have you felt that you were unable to control the important things in your life, in the last month, how often have you felt nervous and stressed, in the last month, how often have you found that you could not cope with all the things that you had to do, in the last month, how often have you felt difficulties were piling up so high that you could not overcome them, in the last month, how often have you felt confident about your ability to handle your personal problems, in the last month, how often have you felt that things were going your way, in the last month, how often have you been able to control irritations in your life, in the last month, how often have you felt that you were on top of things and the like. The method 300 includes obtaining one or more responses of the one or more users on the outputted relaxation questionnaire from the one or more user devices 102. For example, the one or more responses may include never, almost never, sometimes, fairly often, very often and the like. In an exemplary embodiment of the present disclosure, the user may use 1 (never or very rarely true) to 5 (very often or always true) scale to indicate relevance of each statement of the mindfulness questionnaire to the user. For example, when a statement is often true to the user, the user may select ‘4’ and when the statement is sometimes true to the user, the user may select ‘3’. The scoring may also be reversed based on one or more questions in the mindfulness questionnaire. In another exemplary embodiment of the present disclosure, the user may use 1 (very often or always true) to 5 (never or very rarely true) scale to indicate relevance of each statement of the mindfulness questionnaire to the user. Furthermore, the method 300 includes determining one or more relaxation questionnaire scores corresponding to the relaxation questionnaire and one or more relaxation parameters scores for the determined set of wellness parameters corresponding to the relaxation pillar based on the obtained one or more responses, the received request, the predefined wellness information, the set of predefined rules and the predefined pillar weightage by using the trained wellness evaluation based AI model. In an exemplary embodiment of the present disclosure, 10 score is provided for “never” response, 8 score is provided for “almost” response, 6 score is provided for “sometimes” response, 4 score is provided for “fairly often” response and 2 score is provided for “very often” response. The scoring may also be reversed based on one or more questions in the relaxation questionnaire. In another exemplary embodiment of the present disclosure, 2 score is provided for “never” response, 4 score is provided for “almost” response, 6 score is provided for “sometimes” response, 8 score is provided for “fairly often” response and 10 score is provided for “very often” response. In an exemplary embodiment of the present disclosure, the one or more relaxation scores are calculated by using a Perceived Stress Scale (PSS). For example, the PSS scale includes multiple questions to determine feelings and thoughts of the user during the last month. The method 300 includes generating the relaxation score based on the determined one or more relaxation questionnaire scores, the received request, the one or more relaxation parameters scores, the set of predefined rules and the predefined pillar weightage by using the trained wellness evaluation based AI model. In an exemplary embodiment of the present disclosure, the relaxation score ranging from 80-100 is considered as low stress, the relaxation score ranging from 60-80 is considered as moderate stress, the relaxation score ranging from 40-60 is considered as high stress and the relaxation score ranging from 20-40 is considered as high perceived stress.

Furthermore, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the method 300 includes outputting a mindfulness questionnaire on the user interface screens of the one or more user devices 102. In an exemplary embodiment of the present disclosure, the mindfulness questionnaire is a short form of 15-item Five Facet Mindfulness Questionnaire (FFMQ) including multiple facets associated with observing, describing, acting with awareness, non-judging of inner experience, and non-reactivity to inner experience. In an exemplary embodiment of the present disclosure, the mindfulness questionnaire includes multiples mindfulness questions to determine a mindfulness score. For example, the multiple mindfulness questions include I don't pay attention to what I'm doing because I'm daydreaming, worrying, or otherwise distracted, I believe some of my thoughts are abnormal or bad and I shouldn't think that way, I have trouble thinking of the right words to express how I feel about things, I do jobs or tasks automatically without being aware of what I'm doing, I think some of my emotions are bad or inappropriate and I shouldn't feel them, I find myself doing things without paying attention, I tell myself I shouldn't be feeling the way I'm feeling, when I take a shower or a bath, I stay alert to the sensations of water on my body, I'm good at finding words to describe my feelings, when I have distressing thoughts or images, I “step back” and am aware of the thought or image without getting taken over by it, I notice how foods and drinks affect my thoughts, bodily sensations, and emotions, when I have distressing thoughts or images I am able just to notice them without reacting, I pay attention to sensations, such as the wind in my hair or sun on my face, even when I'm feeling terribly upset I can find a way to put it into words, when I have distressing thoughts or images I just notice them and let them go and the like. The method 300 includes obtaining one or more responses of the one or more users on the outputted mindfulness questionnaire from the one or more user devices 102. For example, the one or more responses may include never, almost never, sometimes, fairly often, very often and the like. Further, the method 300 includes determining one or more mindfulness questionnaire scores corresponding to the mindfulness questionnaire and one or more mindfulness parameters scores for the determined set of wellness parameters corresponding to the mindfulness pillar based on the obtained one or more responses, the received request, the predefined wellness information, the set of predefined rules and the predefined pillar weightage by using the trained wellness evaluation based AI model. In an exemplary embodiment of the present disclosure, the set of wellness parameters corresponding to the mindfulness pillar include calm time, focus time, training time and the like. Furthermore, the method 300 includes generating a mindfulness score based on the determined one or more mindfulness questionnaire scores, the received request, the one or more mindfulness parameters scores, the set of predefined rules and the predefined pillar weightage by using the trained wellness evaluation based AI model.

Further, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the method 300 includes determining one or more nutrition parameters scores for the determined set of wellness parameters corresponding to the nutrition pillar based on the received request, the set of predefined rules and the predefined pillar weightage by using the trained wellness evaluation based AI model. In an embodiment of the present disclosure, the set of wellness parameters corresponding to the nutrition pillar are obtained via a health device 108. In an exemplary embodiment of the present disclosure, the health device may be a finger prick device. The health device 108 may be a wearable device. In another embodiment of the present disclosure, the set of wellness parameters corresponding to the nutrition pillar are obtained via a collection of bodily matter, such as stool, urine and the like. In an exemplary embodiment of the present disclosure, the set of wellness parameters corresponding to the nutrition pillar comprise: Body Mass Index (BMI), glucose, total cholesterol, risk ratio, Low-Density Lipoprotein (LDL), High-Density Lipoprotein (HDL), triglycerides, gut microbiome analysis, stress analysis, immune system health, biological age and the like. For example, the set of predefined rules corresponding to the nutrition pillar are: BMI=weight divided by height squared=over 25 BMI no points Vs under 25 BMI=16.67 points, glucose=non fasting under 140 fasting under 100=16.67 points, total cholesterol—in normal range add full credit, if over range no points are added under 200=16.67 points, LDL—under 100=16.67 points, HDL—over 60=16.67 points, triglycerides—less than 150=16.67 points and the like. Furthermore, the method 300 includes generating a nutrition score based on the determined one or more nutrition parameter scores for the determined set of wellness parameters, the received request, the set of predefined rules and the predefined pillar weightage by using the trained wellness evaluation based AI model. In an exemplary embodiment of the present disclosure, when the nutrition pillar includes six set of wellness parameters, each of the one or more nutrition parameters scores for the six set of wellness parameters may be 16.67.

In an embodiment of the present disclosure, in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the method 300 includes determining one or more sleep parameters scores for the determined set of wellness parameters corresponding to the sleep pillar based on the received request, the set of predefined rules and the predefined pillar weightage by using the trained wellness evaluation based AI model. In an exemplary embodiment of the present disclosure, the set of wellness parameters corresponding to the sleep pillar include total time in bed, sleep latency, readiness, activity, sleep waking, actual sleep time, wakefulness, sleep efficiency, efficiency resting heart rate, Heart Rate Variability (HRV), respiration rate, body temperature and the like. For example, the set of predefined rules for calculating sleep efficiency is 480 (total minutes in bed)−30 (minutes to fall asleep)−0 (minutes awake during the night)=450 (actual sleep time in minutes) i.e., 450/480=0.9375×100=93.75% sleep efficiency. Furthermore, the method 300 includes generating a sleep score based on the determined one or more sleep parameter scores for the determined set of wellness parameters, the received request, the set of predefined rules and the predefined pillar weightage by using the trained wellness evaluation based AI model.

In an embodiment of the present disclosure, each of the set of wellness parameters corresponding to the one or more wellness pillars are categorized into one or more wellness categories based on a set of parameter scores. In an exemplary embodiment of the present disclosure, the one or more relaxation categories include elite, advanced, intermediate, beginner, new and the like. In an exemplary embodiment of the present disclosure, the set of parameter scores include the one or more fitness parameters scores, the one or more relaxation questionnaire scores, the one or more relaxation parameters scores, the one or more mindfulness questionnaire scores, the one or more mindfulness parameters scores, the one or more nutrition parameters scores and the one or more sleep parameters scores.

At step 308, a wellness score of the one or more users is generated based on the generated pillar score of each of the one or more wellness pillars, the received request, the set of predefined rules and the predefined pillar weightage by using the trained wellness evaluation based AI model. In generating the wellness score of the one or more users based on the generated pillar score of each of the one or more wellness pillars, the received request, the set of predefined rules and the predefined pillar weightage by using the trained wellness evaluation based AI model, the method 300 includes correlating the pillar score of each of the one or more wellness pillars, the received request, the set of predefined rules and the predefined pillar weightage by using the trained wellness evaluation based AI model. In an embodiment of the present disclosure, the pillar score of each of the one or more wellness pillars include the fitness score, the relaxation score, the nutrition score, the mindfulness score and the sleep score. Further, the method 300 includes generating the wellness score of the one or more users based on the result of correlation.

At step 310, level of wellness of the one or more users is determined based on the generated wellness score, predefined wellness information and the received request by using the trained wellness evaluation based AI model. In an exemplary embodiment of the present disclosure, the level of wellness of the one or more users include elite, advanced, intermediate, beginner, new and the like.

At step 312, the determined set of wellness parameters corresponding to each of the one or more wellness pillars, the generated pillar score for each of the one or more wellness pillars, the generated wellness score and the determined level of wellness are outputted on user interface screens of the one or more user devices 102. In an exemplary embodiment of the present disclosure, the determined set of wellness parameters corresponding to each of the one or more wellness pillars, the generated pillar score for each of the one or more wellness pillars, the generated wellness score and the determined level of wellness are outputted on the user interface screens of the one or more user devices 102 by using Transport layer Security (TLS) 1.2.

Further, the method 300 includes receiving one or more wellness preferences from the one or more user devices 102. In an exemplary embodiment of the present disclosure, the one or more wellness preferences include weight loss, weight gain, stress management, anxiety management, sleep management and the like. Further, the method 300 includes dynamically allocating one or more parameter weightages to the set of wellness parameters of each of the one or more wellness pillars based on the received one or more wellness preferences, the received request and the predefined wellness information by using the trained wellness evaluation based AI model. The one or more parameter weightages include one or more maximum parameter scores for the set of wellness parameters of each of the one or more wellness pillars. In an exemplary embodiment of the present disclosure, each of the one or more parameter weightages may not be less than 1. In an embodiment of the present disclosure, the set of parameter scores for the set of wellness parameters corresponding to each of the one or more wellness pillars are generated based on the allocated one or more parameter weightages. The set of parameter scores for the set of wellness parameters may be equal or less than the one or more maximum parameter scores. Furthermore, the method 300 includes dynamically allocating a pillar weightage to each of the one or more wellness pillars based on the received one or more wellness preferences, the received request and the predefined wellness information by using the trained wellness evaluation based AI model. In an embodiment of the present disclosure, the pillar weightage includes maximum pillar score of each of the one or more wellness pillars. In an exemplary embodiment of the present disclosure, combined pillar weightage of the one or more wellness pillar may not exceed 100. In another exemplary embodiment of the present disclosure, the combined pillar weightage of the one or more wellness pillar may exceed 100. In an embodiment of the present disclosure, the pillar score for each of the one or more wellness pillars is generated based on the allocated pillar weightage. The pillar score for each of the one or more wellness pillars may be equal or less than the maximum pillar score. In an exemplary embodiment of the present disclosure, the one or more parameter weightages and the pillar weightage are in percentage form. In an embodiment of the present disclosure, the set of parameter scores generated based on the one or more parameter weightages of a specific pillar may again be quantified based on the pillar weightage of the specific pillar to generate the pillar score.

In an embodiment of the present disclosure, the method 300 includes receiving the pillar weightage for each of the one or more wellness pillars and the one or more parameter weightages for the set of wellness parameters of each of the one or more wellness pillars from the one or more user devices 102. In another embodiment of the present disclosure, the pillar weightage for each of the one or more wellness pillars may be equally distributed. For example, the sleep pillar has the pillar weightage of 20, the nutrition pillar has the pillar weightage of 20, the fitness pillar has the pillar weightage of 20, the mindfulness pillar has the pillar weightage of 20 and the relaxation pillar has the pillar weightage of 20. Further, combination of the one or more parameter weightages corresponding to each of the one or more wellness pillars may not exceed the pillar weightage of each of the one or more wellness pillars. For example, if the pillar weightage of the sleep pillar is 20, then the combination of the one or more parameter weightages for the set of wellness parameters of the sleep pillar, such as sleep, readiness, heart rate variability, activity and the like may not exceed 20. In an embodiment of the present disclosure, the one or more parameter weightages for the set of wellness parameters may be equally distributed. The method 300 includes allocating the received pillar weightage to each of the one or more wellness pillars. Furthermore, the method 300 includes allocating the received one or more parameter weightages to the set of wellness parameters of each of the one or more wellness pillars.

Furthermore, the method 300 includes determining if the determined level of wellness of the one or more users is below a predefined threshold wellness level. Further, the method 300 includes determining one or more root causes for the determined level of wellness based on the determined level of wellness, the set of parameter scores and the predefined wellness information by using the trained wellness evaluation based AI model upon determining that the determined level of wellness is below the predefined threshold wellness level. In an exemplary embodiment of the present disclosure, the one or more root causes may be less sleep, high cholesterol, high sugar, high blood pressure and the like. The method 300 includes predicting one or more possible health conditions of the one or more users based on the determined one or more root causes, the determined level of wellness, the set of parameter scores and the predefined wellness information by using the trained wellness evaluation based AI model. For example, the one or more possible health conditions may be heart attack, diabetes and the like. Furthermore, the method 300 includes predicting time of occurrence of the predicted one or more possible conditions based on the determined one or more root causes, the determined level of wellness, the set of parameter scores and the predefined wellness information by using the trained wellness evaluation based AI model. In an embodiment of the present disclosure, the determined one or more root causes, the predicted one or more possible health conditions and the predicted time of occurrence of the predicted one or more possible conditions are outputted on the user interface screens of the one or more user devices 102.

The method 300 may be implemented in any suitable hardware, software, firmware, or combination thereof.

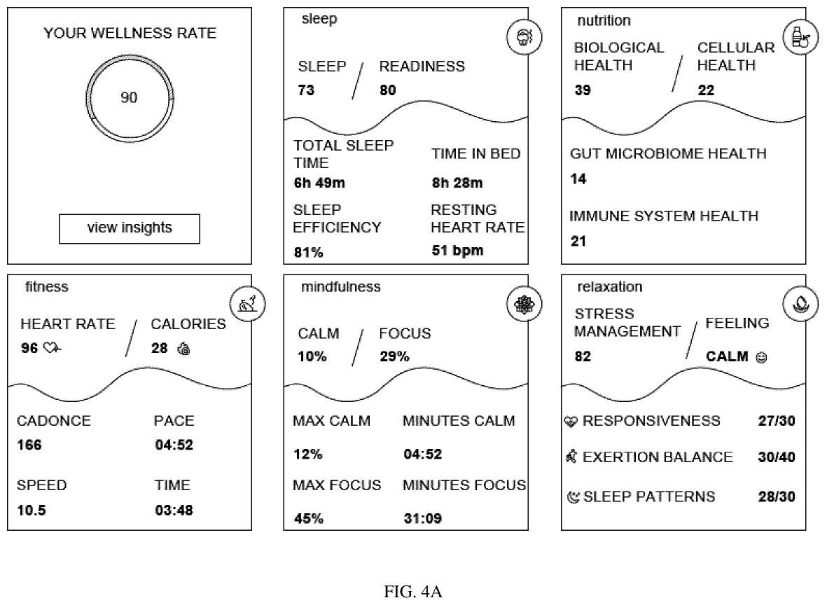

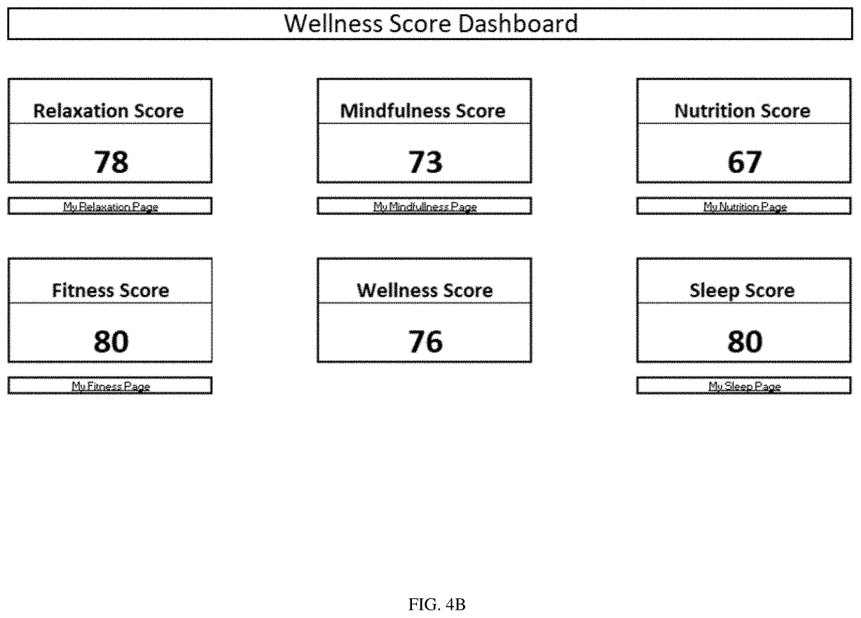

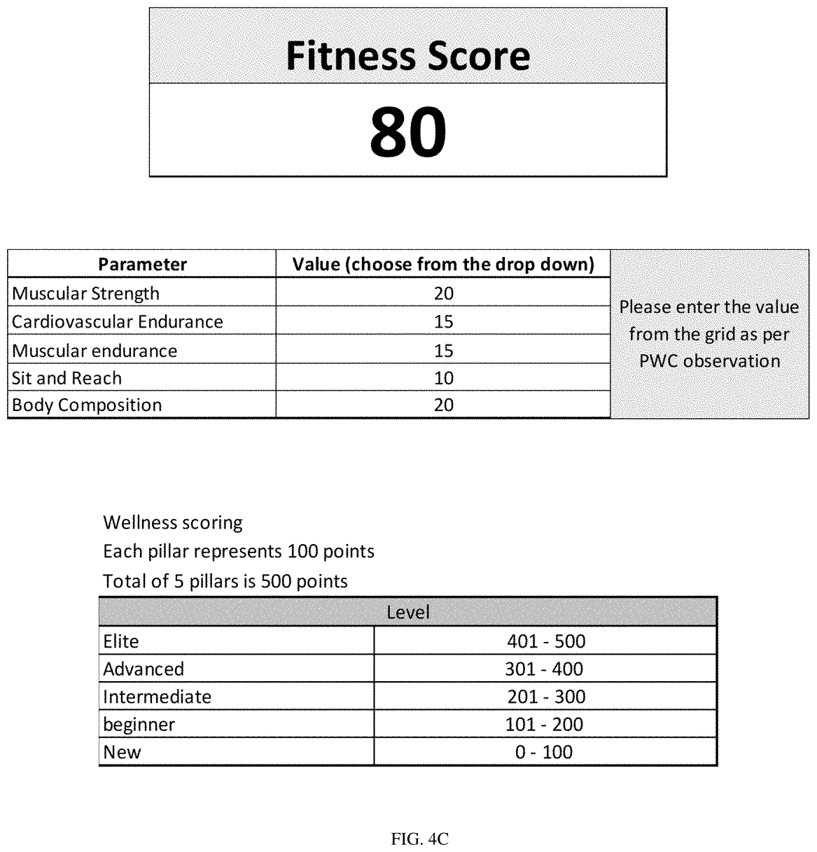

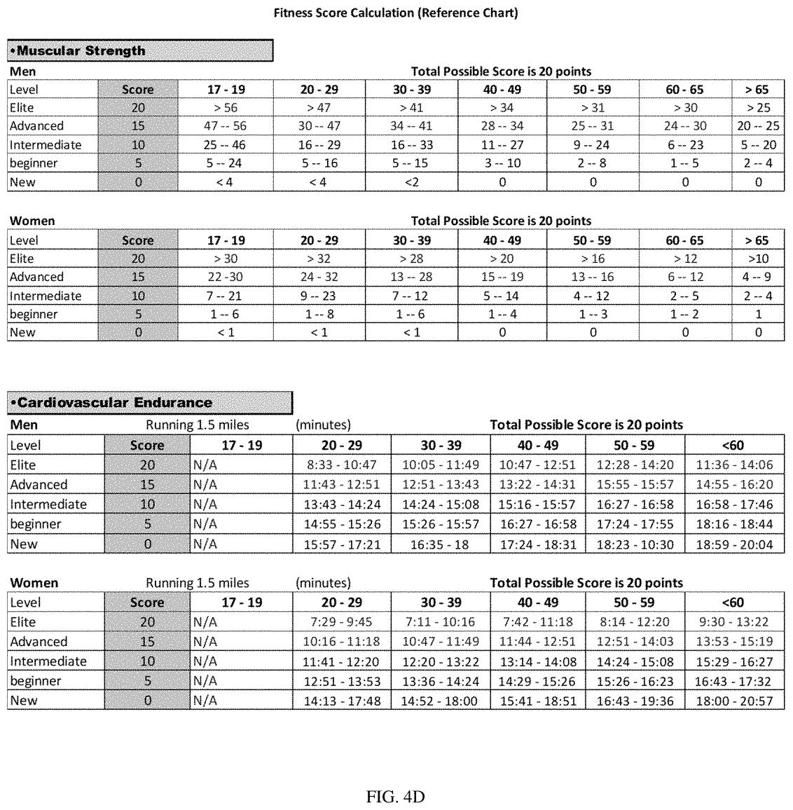

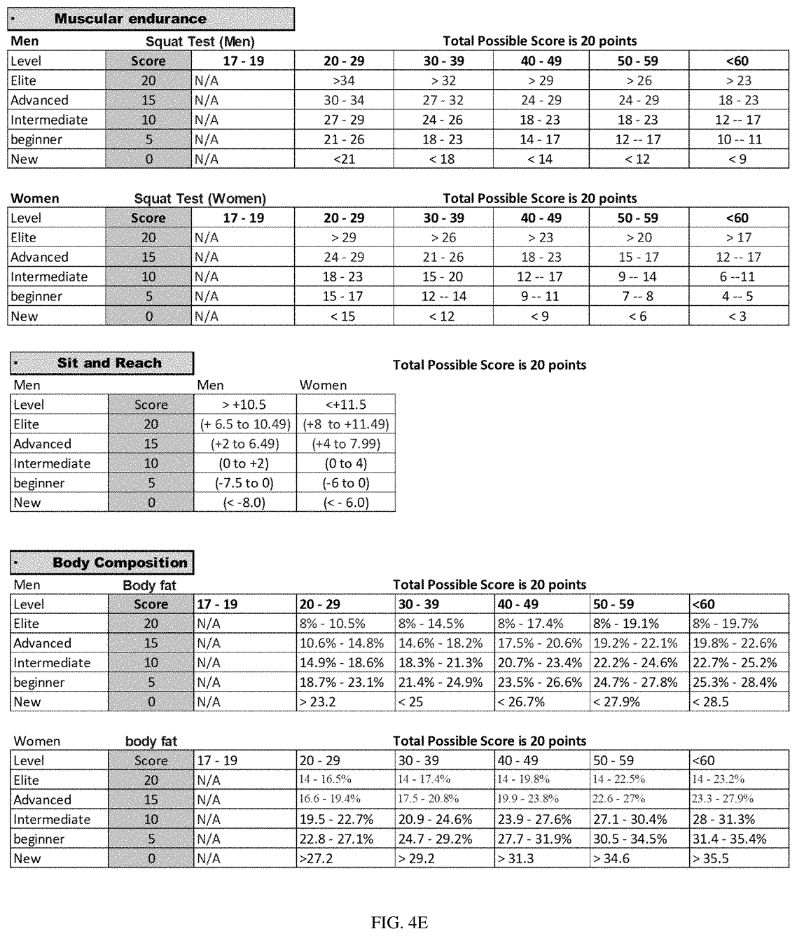

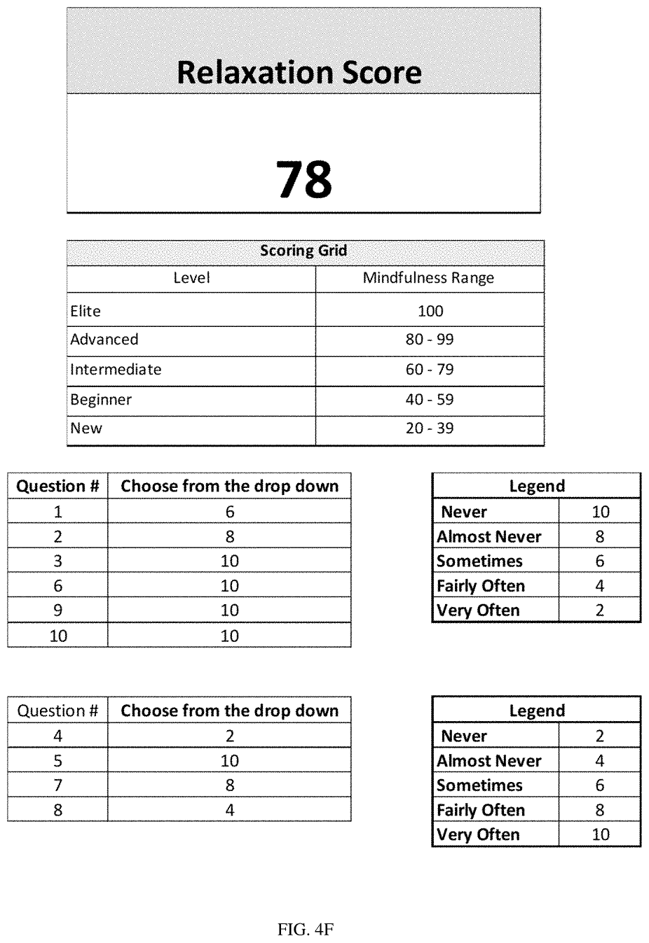

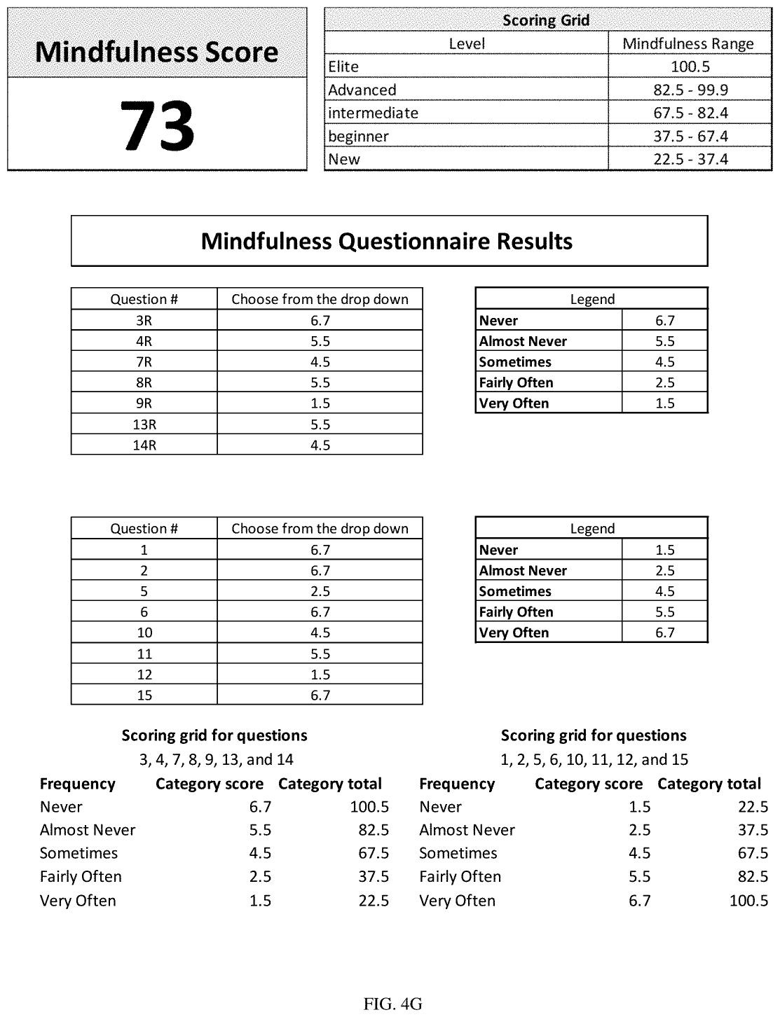

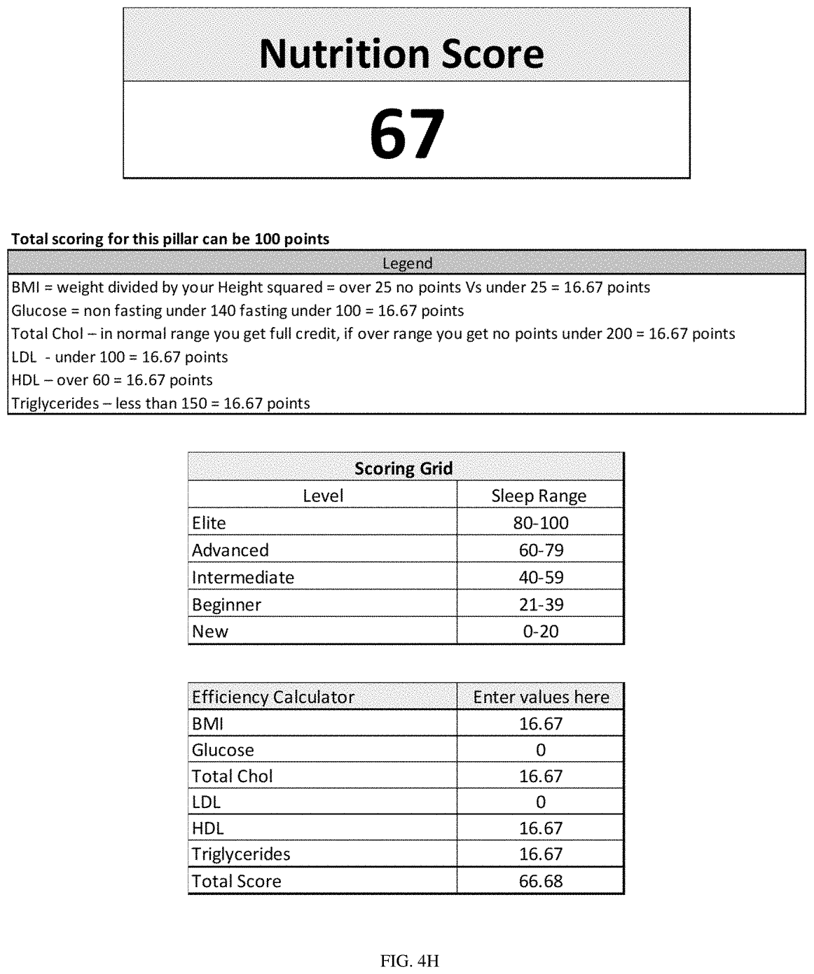

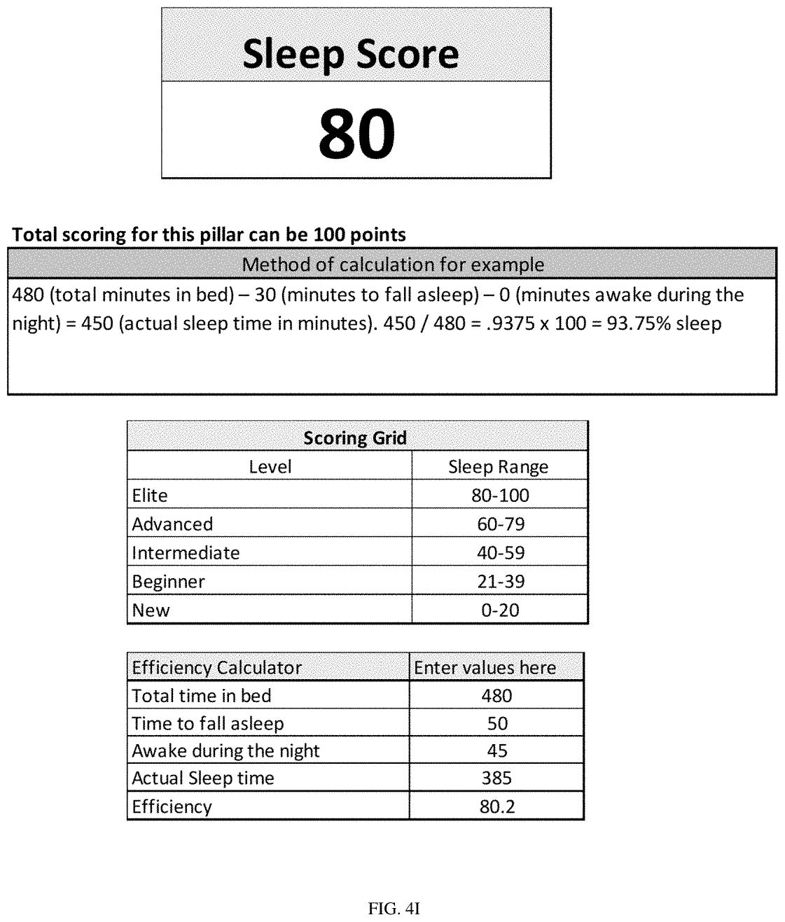

FIGS. 4A-4I are graphical user interface screens of dashboard of the computing system 104 for evaluating wellness of the one or more users, in accordance with an embodiment of the present disclosure. FIG. 4A is a dashboard of the computing system 104. The dashboard shows wellness rate of the user, sleep and readiness score along with total sleep time, time in bed, sleep efficiency and resting heart rate, biological health and cellular health score along with gut microbiome health score and immune system health score, heart rate and calories burnt along with cadence, pace, speed and time, calm and focus percentage along with max calm percentage, minutes calm, max focus percentage and minutes focus and stress management score and kind of feeling along with responsiveness, exertion balance and sleep patterns. In the current scenario, the kind of feeling is calm. Further, FIG. 4B is the dashboard of the computing system 104 displaying relaxation score, mindfulness score, nutrition score, fitness score, wellness score and sleep score. In an embodiment of the present disclosure, the one or more users may also access various pages of the dashboard by selecting multiple tabs present at bottom of the dashboard, as shown in FIG. 4B. In an exemplary embodiment of the present disclosure, the multiple tabs include wellness score, fitness scoring, relaxation scoring, mindfulness scoring, nutrition scoring and sleep scoring. FIGS. 4C-4E are graphical user interface screens for fitness scoring. The graphical user interface screens depict the set of wellness parameters corresponding to the fitness pillar, such as muscle strength, cardiovascular endurance, muscular endurance, sit and reach, body composition and the like. The set of parameter scores corresponding to the fitness pillar are generated in accordance with age groups, experience level, gender of the one or more users and the like. FIG. 4F is a graphical user interface screen for relaxation scoring. The one or more users are required to provide the one or more responses to relaxation questionnaires in accordance with the legend tables, as shown in FIG. 4F. Similarly, FIG. 4G is a graphical user interface screen for mindfulness scoring. In an exemplary embodiment of the present disclosure, the mindfulness questionnaire may include multiple questions including observing questions, describing questions, acting with awareness questions, non-judging questions, non-reactivity questions, reversed-phrased questions and the like. FIG. 4H is a graphical user interface screen for nutrition scoring. Similarly, FIG. 4I is a graphical user interface screen for sleep scoring.

Thus, various embodiments of the present computing system 104 provide a solution to evaluate wellness of the one or more users. Since, the computing system 104 considers the one or more wellness pillars including relaxation pillar, fitness pillar, mindfulness pillar, nutrition pillar and sleep pillar while generating the wellness score, the computing system 104 is accurate and precise. Further, the computing system 104 predicts the one or more possible health conditions, such as heart attack, diabetes and the like, of the one or more users and time of occurrence of the one or more possible health conditions. Thus, the one or more users may receive early treatment and change their lifestyle to allay or prevent the occurrence of the one or more possible health conditions.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

1. A computing system for evaluating wellness of one or more users, the computing system comprising:
one or more virtualized hardware processors; and
a memory coupled to the one or more virtualized hardware processors, wherein the memory comprises a plurality of modules in the form of programmable instructions executable by the one or more virtualized hardware processors, wherein the plurality of modules comprises:
a request receiver module configured to receive a request from one or more user devices to evaluate wellness of one or more users, wherein the request comprises: name, address, weight, height, glucose, cholesterol, triglycerides, gender, age and experience level of the one or more users;
a parameter determination module configured to determine a set of wellness parameters corresponding to each of one or more wellness pillars based on the received request and a set of predefined rules by using a trained wellness evaluation based Artificial Intelligence (AI) model, wherein the one or more wellness pillars comprise: relaxation pillar, fitness pillar, mindfulness pillar, nutrition pillar and sleep pillar;
a pillar score generation module configured to generate a pillar score for each of the one or more wellness pillars based on the received request, the set of predefined rules, the determined set of wellness parameters corresponding to each of the one or more wellness pillars and a predefined pillar weightage by using the trained wellness evaluation based AI model;
a wellness score generation module configured to generate a wellness score of the one or more users based on the generated pillar score of each of the one or more wellness pillars, the received request, the set of predefined rules and the predefined pillar weightage by using the trained wellness evaluation based AI model;
a wellness level determination module configured to determine level of wellness of the one or more users based on the generated wellness score, predefined wellness information and the received request by using the trained wellness evaluation based AI model; and
a data output module configured to output the determined set of wellness parameters corresponding to each of the one or more wellness pillars, the generated pillar score for each of the one or more wellness pillars, the generated wellness score and the determined level of wellness on user interface screens of the one or more user devices.

2. The computing system of claim 1, wherein in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module is configured to:
determine one or more fitness parameters scores for the determined set of wellness parameters corresponding to the fitness pillar based on the received request, the set of predefined rules and the predefined pillar weightage by using the trained wellness evaluation based AI model, wherein the set of wellness parameters corresponding to the fitness pillar comprise: muscular strength, cardiovascular endurance, muscular endurance, flexibility, sit and reach, body composition, calories, cadence, distance, pace, heart rate and duration; and
generate a fitness score based on the determined one or more fitness parameters scores for the determined set of wellness parameters, the received request, the set of predefined rules and the predefined pillar weightage by using the trained wellness evaluation based AI model.

3. The computing system of claim 1, wherein in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module is configured to:
output a relaxation questionnaire on the user interface screens of the one or more user devices;
obtain one or more responses of the one or more users on the outputted relaxation questionnaire from the one or more user devices;
determine one or more relaxation questionnaire scores corresponding to the relaxation questionnaire and one or more relaxation parameters scores for the determined set of wellness parameters corresponding to the relaxation pillar based on the obtained one or more responses, the received request, the predefined wellness information, the set of predefined rules and the predefined pillar weightage by using the trained wellness evaluation based AI model; and
generate a relaxation score based on the determined one or more relaxation questionnaire scores, the received request, the one or more relaxation parameters scores, the set of predefined rules and the predefined pillar weightage by using the trained wellness evaluation based AI model.

4. The computing system of claim 1, wherein in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module is configured to:
output a mindfulness questionnaire on the user interface screens of the one or more user devices;
obtain one or more responses of the one or more users on the outputted mindfulness questionnaire from the one or more user devices;
determine one or more mindfulness questionnaire scores corresponding to the mindfulness questionnaire and one or more mindfulness parameters scores for the determined set of wellness parameters corresponding to the mindfulness pillar based on the obtained one or more responses, the received request, the predefined wellness information, the set of predefined rules and the predefined pillar weightage by using the trained wellness evaluation based AI model, wherein the set of wellness parameters corresponding to the mindfulness pillar comprise: calm time, focus time and training time; and
generate a mindfulness score based on the determined one or more mindfulness questionnaire scores, the received request, the one or more mindfulness parameters scores, the set of predefined rules and the predefined pillar weightage by using the trained wellness evaluation based AI model.

5. The computing system of claim 1, wherein in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module is configured to:
determine one or more nutrition parameters scores for the determined set of wellness parameters corresponding to the nutrition pillar based on the received request, the set of predefined rules and the predefined pillar weightage by using the trained wellness evaluation based AI model, wherein the set of wellness parameters corresponding to the nutrition pillar comprise: Body Mass Index (BMI), glucose, total cholesterol, risk ratio, Low-Density Lipoprotein (LDL), High-Density Lipoprotein (HDL), triglycerides, gut microbiome analysis, stress analysis, immune system health and biological age; and
generate a nutrition score based on the determined one or more nutrition parameter scores for the determined set of wellness parameters, the received request, the set of predefined rules and the predefined pillar weightage by using the trained wellness evaluation based AI model.

6. The computing system of claim 1, wherein in generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model, the pillar score generation module is configured to:
determine one or more sleep parameters scores for the determined set of wellness parameters corresponding to the sleep pillar based on the received request, the set of predefined rules and the predefined pillar weightage by using the trained wellness evaluation based AI model, wherein the set of wellness parameters corresponding to the sleep pillar comprise: total time in bed, sleep latency, readiness, activity, sleep waking, actual sleep time, wakefulness, sleep efficiency, efficiency resting heart rate, Heart Rate Variability (HRV), respiration rate and body temperature; and
generate a sleep score based on the determined one or more sleep parameter scores for the determined set of wellness parameters, the received request, the set of predefined rules and the predefined pillar weightage by using the trained wellness evaluation based AI model.

7. The computing system of claim 1, wherein in generating the wellness score of the one or more users based on the generated pillar score of each of the one or more wellness pillars, the received request, the set of predefined rules and the predefined pillar weightage by using the trained wellness evaluation based AI model, the wellness score generation module is configured to:
correlate the pillar score of each of the one or more wellness pillars, the received request, the set of predefined rules and the predefined pillar weightage by using the trained wellness evaluation based AI model, wherein the pillar score of each of the one or more wellness pillars comprises: fitness score, relaxation score, nutrition score, mindfulness score and sleep score; and
generate the wellness score of the one or more users based on the result of correlation.

8. The computing system of claim 5, wherein the set of wellness parameters corresponding to the nutrition pillar are obtained via one of: a health device and a collection of bodily matters, wherein the health device may be a finger prick device.

9. The computing system of claim 1, further comprises a weightage allocation module configured to:
receive one or more wellness preferences from the one or more user devices, wherein the one or more wellness preferences comprise: weight loss, weight gain, stress management, anxiety management and sleep management;
dynamically allocate one or more parameter weightages to the set of wellness parameters of each of the one or more wellness pillars based on the received one or more wellness preferences, the received request and the predefined wellness information by using the trained wellness evaluation based AI model, wherein a set of parameter scores for the set of wellness parameters of each of the one or more wellness pillars are generated based on the allocated one or more parameter weightages and wherein the set of parameter scores comprise: one or more fitness parameters scores, one or more relaxation questionnaire scores, one or more relaxation parameters scores, one or more mindfulness questionnaire scores, one or more mindfulness parameters scores, one or more nutrition parameters scores and one or more sleep parameters scores; and
dynamically allocate a pillar weightage to each of the one or more wellness pillars based on the received one or more wellness preferences, the received request and the predefined wellness information by using the trained wellness evaluation based AI model, wherein the pillar score for each of the one or more wellness pillars is generated based on the allocated pillar weightage.

10. The computing system of claim 1, further comprises a data prediction module configured to:
determine if the determined level of wellness of the one or more users is below a predefined threshold wellness level, wherein the level of wellness of the one or more users comprises: elite, advanced, intermediate, beginner and new;
determine one or more root causes for the determined level of wellness based on the determined level of wellness, set of parameter scores and the predefined wellness information by using the trained wellness evaluation based AI model upon determining that the determined level of wellness is below the predefined threshold wellness level;
predict one or more possible health conditions of the one or more users based on the determined one or more root causes, the determined level of wellness, the set of parameter scores and the predefined wellness information by using the trained wellness evaluation based AI model; and
predict time of occurrence of the predicted one or more possible conditions based on the determined one or more root causes, the determined level of wellness, the set of parameter scores and the predefined wellness information by using the trained wellness evaluation based AI model, wherein the determined one or more root causes, the predicted one or more possible health conditions and the predicted time of occurrence of the predicted one or more possible conditions are outputted on the user interface screens of the one or more user devices.

11. A method for evaluating wellness of one or more users, the method comprising:
receiving, by one or more hardware processors, a request from one or more user devices to evaluate wellness of one or more users, wherein the request comprises: name, address, weight, height, glucose, cholesterol, triglycerides, gender, age and experience level of the one or more users;
determining, by the one or more hardware processors, a set of wellness parameters corresponding to each of one or more wellness pillars based on the received request and a set of predefined rules by using a trained wellness evaluation based Artificial Intelligence (AI) model, wherein the one or more wellness pillars comprise: relaxation pillar, fitness pillar, mindfulness pillar, nutrition pillar and sleep pillar;
generating, by the one or more hardware processors, a pillar score for each of the one or more wellness pillars based on the received request, the set of predefined rules, the determined set of wellness parameters corresponding to each of the one or more wellness pillars and a predefined pillar weightage by using the trained wellness evaluation based AI model;
generating, by the one or more hardware processors, a wellness score of the one or more users based on the generated pillar score of each of the one or more wellness pillars, the received request, the set of predefined rules and the predefined pillar weightage by using the trained wellness evaluation based AI model;
determining, by the one or more hardware processors, level of wellness of the one or more users based on the generated wellness score, predefined wellness information and the received request by using the trained wellness evaluation based AI model;
outputting, by the one or more hardware processors, the determined set of wellness parameters corresponding to each of the one or more wellness pillars, the generated pillar score for each of the one or more wellness pillars, the generated wellness score and the determined level of wellness on user interface screens of the one or more user devices.

12. The method of claim 11, wherein generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model comprises:
determining one or more fitness parameters scores for the determined set of wellness parameters corresponding to the fitness pillar based on the received request, the set of predefined rules and the predefined pillar weightage by using the trained wellness evaluation based AI model, wherein the set of wellness parameters corresponding to the fitness pillar comprise: muscular strength, cardiovascular endurance, muscular endurance, flexibility, sit and reach, body composition, calories, cadence, distance, pace, heart rate and duration; and
generating a fitness score based on the determined one or more fitness parameters scores for the determined set of wellness parameters, the received request, the set of predefined rules and the predefined pillar weightage by using the trained wellness evaluation based AI model.

13. The method of claim 11, wherein generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model comprises:
outputting a relaxation questionnaire on the user interface screens of the one or more user devices;
obtaining one or more responses of the one or more users on the outputted relaxation questionnaire from the one or more user devices;
determining one or more relaxation questionnaire scores corresponding to the relaxation questionnaire and one or more relaxation parameters scores for the determined set of wellness parameters corresponding to the relaxation pillar based on the obtained one or more responses, the received request, the predefined wellness information, the set of predefined rules and the predefined pillar weightage by using the trained wellness evaluation based AI model; and
generating a relaxation score based on the determined one or more relaxation questionnaire scores, the received request, the one or more relaxation parameters scores, the set of predefined rules and the predefined pillar weightage by using the trained wellness evaluation based AI model.

14. The method of claim 11, wherein generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model comprises:
outputting a mindfulness questionnaire on the user interface screens of the one or more user devices;
obtaining one or more responses of the one or more users on the outputted mindfulness questionnaire from the one or more user devices;
determining one or more mindfulness questionnaire scores corresponding to the mindfulness questionnaire and one or more mindfulness parameters scores for the determined set of wellness parameters corresponding to the mindfulness pillar based on the obtained one or more responses, the received request, the predefined wellness information, the set of predefined rules and the predefined pillar weightage by using the trained wellness evaluation based AI model, wherein the set of wellness parameters corresponding to the mindfulness pillar comprise: calm time, focus time and training time; and
generating a mindfulness score based on the determined one or more mindfulness questionnaire scores, the received request, the one or more mindfulness parameters scores, the set of predefined rules and the predefined pillar weightage by using the trained wellness evaluation based AI model.

15. The method of claim 11, wherein generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model comprises:
determining one or more nutrition parameters scores for the determined set of wellness parameters corresponding to the nutrition pillar based on the received request, the set of predefined rules and the predefined pillar weightage by using the trained wellness evaluation based AI model, wherein the set of wellness parameters corresponding to the nutrition pillar comprise: Body Mass Index (BMI), glucose, total cholesterol, risk ratio, Low-Density Lipoprotein (LDL), High-Density Lipoprotein (HDL), triglycerides, gut microbiome analysis, stress analysis, immune system health and biological age; and
generating a nutrition score based on the determined one or more nutrition parameter scores for the determined set of wellness parameters, the received request, the set of predefined rules and the predefined pillar weightage by using the trained wellness evaluation based AI model.

16. The method of claim 11, wherein generating the pillar score for each of the one or more wellness pillars based on the received request, the set of predefined rules, the determined set of wellness parameters corresponding to each of the one or more wellness pillars and the predefined pillar weightage by using the trained wellness evaluation based AI model comprises:
determining one or more sleep parameters scores for the determined set of wellness parameters corresponding to the sleep pillar based on the received request, the set of predefined rules and the predefined pillar weightage by using the trained wellness evaluation based AI model, wherein the set of wellness parameters corresponding to the sleep pillar comprise: total time in bed, sleep latency, readiness, activity, sleep waking, actual sleep time, wakefulness, sleep efficiency, efficiency resting heart rate, Heart Rate Variability (HRV), respiration rate and body temperature; and
generating a sleep score based on the determined one or more sleep parameter scores for the determined set of wellness parameters, the received request, the set of predefined rules and the predefined pillar weightage by using the trained wellness evaluation based AI model.

17. The method of claim 11, wherein generating the wellness score of the one or more users based on the generated pillar score of each of the one or more wellness pillars, the received request, the set of predefined rules and the predefined pillar weightage by using the trained wellness evaluation based AI model comprises:
correlating the pillar score of each of the one or more wellness pillars, the received request, the set of predefined rules and the predefined pillar weightage by using the trained wellness evaluation based AI model, wherein the pillar score of each of the one or more wellness pillars comprises: fitness score, relaxation score, nutrition score, mindfulness score and sleep score; and
generating the wellness score of the one or more users based on the result of correlation.

18. The method of claim 15, wherein the set of wellness parameters corresponding to the nutrition pillar are obtained via one of: a health device and a collection of bodily matters, wherein the health device may be a finger prick device.

19. The method of claim 11, further comprises:
receiving one or more wellness preferences from the one or more user devices, wherein the one or more wellness preferences comprise: weight loss, weight gain, stress management, anxiety management and sleep management;
dynamically allocating one or more parameter weightages to the set of wellness parameters of each of the one or more wellness pillars based on the received one or more wellness preferences, the received request and the predefined wellness information by using the trained wellness evaluation based AI model, wherein a set of parameter scores for the set of wellness parameters of each of the one or more wellness pillars are generated based on the allocated one or more parameter weightages and wherein the set of parameter scores comprise: one or more fitness parameters scores, one or more relaxation questionnaire scores, one or more relaxation parameters scores, one or more mindfulness questionnaire scores, one or more mindfulness parameters scores, one or more nutrition parameters scores and one or more sleep parameters scores; and
dynamically allocating a pillar weightage to each of the one or more wellness pillars based on the received one or more wellness preferences, the received request and the predefined wellness information by using the trained wellness evaluation based AI model, wherein the pillar score for each of the one or more wellness pillars is generated based on the allocated pillar weightage.

20. The method of claim 11, further comprises:
determining if the determined level of wellness of the one or more users is below a predefined threshold wellness level, wherein the level of wellness of the one or more users comprises: elite, advanced, intermediate, beginner and new;
determining one or more root causes for the determined level of wellness based on the determined level of wellness, set of parameter scores and the predefined wellness information by using the trained wellness evaluation based AI model upon determining that the determined level of wellness is below the predefined threshold wellness level;
predicting one or more possible health conditions of the one or more users based on the determined one or more root causes, the determined level of wellness, the set of parameter scores and the predefined wellness information by using the trained wellness evaluation based AI model; and
predicting time of occurrence of the predicted one or more possible conditions based on the determined one or more root causes, the determined level of wellness, the set of parameter scores and the predefined wellness information by using the trained wellness evaluation based AI model, wherein the determined one or more root causes, the predicted one or more possible health conditions and the predicted time of occurrence of the predicted one or more possible conditions are outputted on the user interface screens of the one or more user devices.
